(19) United States
(12) Patent Application Publication
(10) Pub. No.: US 2011/0200662 A1
(43) Pub. Date: Aug. 18, 2011

(54) Method For The Treatment Of Proliferative Disorders Of The Eye

(76) Inventor: Arnold Glazier, Newton, MA (US)

(21) Appl. No.: 13/125,536

(22) PCT Filed: Oct. 22, 2009
(86) PCT No.: PCT/US09/05744

§ 371 Date: Apr. 21, 2011

Related U.S. Application Data
(60) Provisional application No. 61/196,894, filed on Oct. 22, 2008.

(51) Int. Cl. A61K 31/409; A61K 31/407; A61K 9/00; A61P 27/02

(52) U.S. Cl. 424/429; 514/411

(57) ABSTRACT

The present invention relates to method for the treatment or prevention and of proliferative eye diseases including but not limited to: age related macular degeneration associated proliferative retinopathy, proliferative diabetic retinopathy, proliferative vitreoretinopathy, posterior capsular opacification, scaring and fibrosis after glaucoma filtration surgery, uveal melanoma, and retinoblastoma. The method comprises contacting cells in the eye by means of intra-ocular injection or infusion, with a drug that irreversibly inhibits cellular proliferation without causing extensive tissue necrosis or cytotoxicity. In a preferred embodiment the drug is bizelesin.

### RELATED APPLICATION

This application claims the benefit of U.S. Provisional Application No. 61/196,894, filed on Oct. 22, 2008. The entire teachings of the above application are incorporated herein by reference.

### BACKGROUND OF THE INVENTION

Proliferative diseases of the eye are the leading cause of blindness and vision loss in the United States. Proliferation of blood vessels and scar tissue within the eye plays a major role in a number of diseases that result in vision loss, including age-related macular degeneration (ARMD), proliferative diabetic retinopathy (PDR), and proliferative vitreoretinopathy (PVR). Approximately 1.8 million Americans have severe ARMD and 4 million people in the U.S. have diabetic retinopathy. These numbers are expected to nearly double by the year 2020. PVR develops in approximately 8% of patients who have surgery for retinal detachment.

ARMD proliferative retinopathy is characterized by choroidal neovascularization, vascular leakage, fibrosis, macular atrophy and vision loss. PDR is characterized by new blood vessel growth anterior to the retina, bleeding, proliferation of inflammatory cells and fibrosis, which can ultimately lead to retinal detachment and blindness. PVR involves the proliferation of retinal-pigmented epithelial cells (RPE) and Muller cells that can lead to the formation of a membrane anterior to the retina and sub-retinal fibrosis. The pre-retinal membrane can cause retinal detachment and also compromise the success of retinal re-attachment surgery.

The drugs, LUCENTIS® (ranibizumab), MACUGEN® (pegaptanib), and AVASTIN® (bevacizumab) block vascular endothelial growth factor (VEGF) and inhibit angiogenesis in the eye. However, these drugs do not inhibit the proliferation of fibroblasts, glial cells, and RPE cells and do not effectively prevent fibrosis and scar tissue formation. In long-term follow-up of patients treated with the VEGF inhibitor, LUCENTIS®, 50% of patients developed retinal fibrosis. (See: Heier, J. S., Retina, 29(6 Suppl): S39-41 (June 2009); and Friedlander, M., J. Clin. Invest., 117(3): 576-86 (March 2007).

In addition, the current drugs generally require intravitreal injections approximately every 1-3 months. While these drugs are beneficial, vision loss can still occur from proliferative processes.

Multiple growth factors contribute to angiogenesis, fibrosis, and pathological proliferative processes in the eye. These growth factors include vascular endothelial growth factor(s), platelet derive growth factor(s), erythropoietin, spingosphine-1-phosphate (SIP-1), transforming growth factor beta-2 (TGF-b), connective tissue growth factor (CTGF), hepatocyte growth factor, insulin-like growth factor I (IGF-1), angiopoietin-2 (Ang-2), basic fibroblast derived growth factor (bFGF), tumor necrosis factor-alpha (TNF), stromal cell-derived factor-1 (SDF-1), and placental growth factor. Blocking all these important growth factors would require the repeated co-administration of multiple drugs over prolonged periods of time.

Posterior capsular opacification (PCO), is a disorder that develops in approximately 10% of patients within a year after cataract surgery. PCO arises from the proliferation of lens epithelial cells and fibroblasts on the lens capsule. POCO can result in vision loss requiring operative treatment. A variety of cytotoxic agents including mitomycin-C, 5-fluorouracil, colchicine, daunorubicin, and thapsisgargin have been explored as potential drugs to prevent PCO. A device called the Perfect Capsule® has been developed to seal the capsular bag and allow irrigation with drug containing solutions. In addition, implantable lens coated with the cytotoxic drug thapsisgargin have been described. Nonetheless PCO remains a significant clinical problem. (See for example: Wormstone, I. M., Exp Eye Res, 74(3): 337-47 (March 2002); Wormstone, I. M. et al., Exp Eye Res, 88(2): 257-69 (February 2009); Duncan, G. et al., Nat Med, 3(9):1026-8 (September 1997); Walker, T. D., Clin Experiment Ophthalmol, 36(9):883-90 (December 2008).

Glaucoma filtration surgery (GFS) is a type of surgical procedure in which a drainage channel is created for anterior chamber aqueous humor to flow to a subconjunctival filtering bleb or drainage site in order to decrease intraocular pressure. Scarring and fibrosis due to excessive cellular proliferation are the major causes of an unsuccessful outcome with GFS in patients. Cytotoxic drugs such as mitomycin-C, 5-fluorouracil, daunorubicin, taxol, and etoposide can help to prevent post-surgical scarring, but also cause widespread cell death that can result in ocular toxicity. (See for example, Lama, P. J. et al., Survey of Ophthalmology, Volume 48, Issue 3, pp. 314-346 (May-June 2003); Georgoulas, S. et al., Prog Brain Res., 173: 237-54 (2008); Takihara, Y. et al., American Journal of Ophthalmology, Volume 147, Issue 5, pp. 912-918 (May 2009); Dong, H. et al., American Journal of Ophthalmology, Volume 132, Issue 6, pp. 875-880 (December 2001); WuDunn, D. et al., American Journal of Ophthalmology, Volume 134, Issue 4, pp. 521-528 (October 2002); Gedde, S. J. et al., American Journal of Ophthalmology, in Press (August 2009); Palanca-Capistrano, A. M. et al., Ophthalmology, Volume 116, Issue 2, pp. 185-190 (February 2009); Singh, K. et al., Ophthalmology, Volume 107, Issue 12, pp. 2305-2309 (December 2000)).

Malignant proliferative diseases of the eye include: ocular cancers, ocular melanoma. ocular lymphoma, retinoblastoma and metastatic lesions to the eye. Current therapies for malignant diseases of the eye such as uveal melanoma and retinoblastoma fail to consistently cure the cancer and can cause vision loss and ocular damage.

The local administration of drugs directly into the eye and into different anatomic sites within the eye is well known to those skilled in the art of ophthalmology. A wide variety of cytotoxic drugs have been evaluated following intraocular administration for the therapy of proliferative diseases of the eye including: taxol, fluorouracil, daunorubicin, melphan, methotrexate, mitomycin-C, actinomycin C, colchicine, 5-fluorodeoxyuridine, vinblastine sulfate, adriamycin, cytosine arabinoside, 5-fluorouridine 5′-monophosphate. Cytotoxic agents however, work by killing cells, have a low therapeutic index, and can cause cellular and ocular damage. Ribozymes to proliferating cell nuclear antigen, which cause transient inhibition of cell proliferation failed in a clinical trial as a therapy for PVR. The intraocular drug, IMS2186, is cytostatic, does not irreversibly arrest the potential for cell proliferation and needs to be given in a long-lasting depot form. Inhibitors to VEGF have been employed with success as anti-angiogenic agents for the inhibition of endothelial cell proliferation within the eye. However, inhibitors of VEGF have reversible anti-proliferative activity against only a limited number of cell types. In addition, a wide variety of growth factors can circumvent the activity of VEGF inhibitors. Accordingly, there is a need for new approaches to the treatment of proliferative diseases of the eye.

The following references relate to this matter: Pastor, J. C., Surv Ophthalmol, 43(1): 3-18 (July-August 1998); Machemer, R., Invest Ophthalmol Vis Sci, 29(12):1771-83 (December 1988); Pastor, J. C. et al., Prog Retin Eye Res, 21(1):127-44 (January 2002); Steinhorst, U. H. et al., Invest Ophthalmol Vis Sci, 34(5):1753-60 (April 1993); Schwartz, S. G. et al., Exp Diabetes Res. 2007:52487 (2007); Porta, M. et al., Pharmacol Ther, 103(2):167-77 (August 2004); Khan, Z. A. et al., Exp Diabesity Res, 4(4):287-301 (October-December 2003); Robertson, D. M., Am J Ophthalmol, 136(1):161-70 (July 2003); Chintagumpala, M. et al., Oncologist, 12(10):1237-46 (October 2007); Moritera, T. et al., Invest Ophthalmol Vis Sci, 33(11): 3125-30 (October 1992); Wiedemann, P. et al., Am J Ophthalmol, 126(4): 550-9 (October 1998); Kirmani, M. et al., Retina, 3(4):269-72 (1983); Yu, H. G. et al., Korean J Ophthalmol, 11(2): 98-105 (December 1997); Mandava, N. et al., Invest Ophthalmol Vis Sci, 43(10): 3338-48 (October 2002); Schiff, W. M. et al., Arch Ophthalmol, 125(9): 1161-7 (September 2007); Berger, A. S. et al., Invest Ophthalmol Vis Sci, 37(11): 2318-25 (October 1996); Lee, J. J. et al., Invest Ophthalmol Vis Sci, 43(9): 3117-24 (September 2002); Wiedemann, P. et al., Invest Ophthalmol Vis Sci, 26(5): 719-25 (May 1985); Assil, K. K. et al., Invest Ophthalmol Vis Sci, 32(11): 2891-7 (October 1991); Suárez, F. et al., Invest Ophthalmol Vis Sci, 48(8): 3437-40 (August 2007); Van Quill, K. R. et al., Ophthalmology, 112(6): 1151-8 (June 2005); Tsui, J. Y. et al., Invest Ophthalmol Vis Sci, 49(2): 490-6 (February 2008); van Bockxmeer, F. M. et al., Invest Ophthalmol Vis Sci, 26(8): 1140-7 (August 1985); Chen, E. P. et al., Invest Ophthalmol Vis Sci, 33(7):2160-4 (June 1992); Hussain, N. et al., Indian J Ophthalmol, 55(6): 445-50 (November-December 2007); Falkenstein, I. A. et al., Curr Eye Res, 33(7): 599-609 (July 2008); Handa, J. T. et al., Exp Eye Res, 62(6): 689-96 (June 1996); Harbour, J. W. et al., Invest Ophthalmol Vis Sci, 37(9): 1892-8 (August 1996); Sohan Singh Hayreh, Progress in Retinal and Eye Research, Volume 26, Issue 5, pp. 470-485 (September 2007).

### SUMMARY OF THE INVENTION

The present invention is directed to methods of use and compositions of drug formulations that irreversibly inhibit the potential for cell proliferation without killing nonproliferating cells, for treating proliferative diseases of the eye including but not limited to; age-related macular degeneration (ARMD), proliferative diabetic retinopathy (PDR), proliferative vitreoretinopathy (PVR), and posterior capsular opacification.

More specifically, the present invention relates to a method for treatment of proliferative diseases, disorders and conditions of the eye in humans and animals (e.g., mammals). In certain embodiments, the condition is neovascularization of the retina or choroidal neovascularization. In particular embodiments, the methods for treatment include treating a premature newborn subjected to oxygen therapy.

Methods for treatment of a proliferative disease, disorder or condition of the eye, is described herein comprising locally administering a drug into the target space of the eye, wherein said drug irreversibly inhibits the potential for cell proliferation, and wherein said drug is not cytotoxic to nonproliferating cells. In certain embodiments, the drug is bizelesin or adozelesin. In particular embodiments, the dose of the drug is in the range of 0.0001 ng to 10.0 ng. In more particular embodiments, the dose is in the range of 0.001 ng to 10 ng.

In certain aspects, the disease, condition or disorder of the eye is selected from: diabetic proliferative retinopathy, age related macular degeneration, associated proliferative retinopathy, proliferative vitreoretinopathy, sub-retinal fibrosis, polypoidal choroidal vasculopathy, proliferative vitreoretinopathy, epimacular membranes, choroidal neovascularization, neovascularization of the retina, retinopathy of prematurity, neovascularization related to ocular histoplasmosis, retinal hemagioblastoma in von Hippel-Landau syndrome, scarring after glaucoma filtration surgery, uveal melanoma, ocular nevi, retinoblastoma, ocular lymphoma, metastatic cancers to the eye, pre-malignant lesions of the eye dysplastic lesions, pigmented nevi or primary acquired conjunctival melanosis.

Also described is a method for the treatment of proliferative eye disorders is described herein comprising: selecting a pharmaceutical formulation, comprising a drug that irreversibly inhibits the potential for cell replication; defining a target space of the eye; and contacting cells in the target space of the eye with said drug by injecting or infusing the drug directly into the target space of the eye at an effective amount for a sufficient period of time to treat the proliferative disorder; and wherein the quantity of said drug is at dose below that required to produce toxicity. The drug is generally noncytotoxic to nonproliferating cells at concentrations that inhibit the potential for cell proliferation. In particular embodiments, the drug is bizelesin or adozelesin.

In another aspect of the invention, a method for treatment of diabetic proliferative retinopathy comprising administering an intravitreal injection of an effective amount of bizelesin is described. In yet another embodiment, treatment of age related macular degeneration associated proliferative retinopathy, comprising administering an intravitreal injection of bizelesin is described.

In certain embodiments, the present invention relates to a method for the treatment of proliferative diseases and conditions of the eye, including but not limited to diabetic proliferative retinopathy, age related macular degeneration associated proliferative retinopathy, proliferative vitreoretinopathy, posterior capsule opacification, and scarring after glaucoma filtration surgery. In a preferred embodiment the drug is bizelesin. In another preferred embodiment the drug is adozelesin. In a certain embodiment, the posterior capsule of the lens is contacted with an effective amount of bizelesin by means of a physical carrier impregnated with the drug or with the drug absorbed on the surface of the physical carrier. In certain aspects, the physical carrier is an implantable lens.

The present invention also relates to kits comprised of a drug formulated and packaged for intraocular administration. In certain embodiments, the kit comprises a container with bizelesin dissolved in a non-aqueous frozen solvent, and in a second container with a buffered saline diluent. In certain embodiments, the kit further comprises a device to administer a unit dose of the drug formulation and concentration wherein drug formulation and concentration are selected such that the desired therapeutic dose and concentration are delivered by the device In certain aspects, the device is a syringe and needle.

Also described is a method for treating proliferative retinopathy comprising administering intravitreal injection of an effective amount of bizelesin wherein the dose is in the range of 0.01 ng to 1.0 ng is described.

In another embodiment, a method for treating proliferative retinopathy comprising administering an effective amount of an intravitreal injection of Bizelesin wherein the dose is in the range of 1.0 ng to 100 ng is described.

The invention also relates to a method for treating posterior capsular opacification comprising administering an effective amount of bizelesin into the capsular bag at the time of cataract surgery.

In certain embodiment, the use of a drug for treating a proliferative disease, disorder or condition of the eye, is described herein comprising locally administering a drug into the target space of the eye, wherein said drug irreversibly inhibits the potential for cell proliferation, and wherein said drug is not cytotoxic to nonproliferating cells. In other embodiments, the manufacture of a medicament for use in treating a proliferative disease, disorder or condition of the eye, is described herein comprising locally administering a drug into the target space of the eye, wherein said drug irreversibly inhibits the potential for cell proliferation, and wherein said drug is not cytotoxic to nonproliferating cells is described.

### BRIEF DESCRIPTION OF THE DRAWINGS

The foregoing will be apparent from the following more particular description of example embodiments of the invention, as illustrated in the accompanying drawings in which like reference characters refer to the same part throughout the different views. The drawings are not necessarily to scale, emphasis instead being placed upon illustrating embodiments of the present invention.

FIG. 1 summarizes the screening results obtained with forty different anticancer agents in the National Cancer Institute (NCI) DTP Human Tumor Cell Line Screen in which approximately 60 different human cancer cell lines were incubated for 48 hours with the anticancer drugs. A colorimetric assay was employed to enable the calculation of the drug concentrations that completely inhibited cell growth (TGI) and the concentrations that resulted in 50% cell killing (LC50). For details on the assay, find on the wide world web at dtp.nci.nih.gov/branches/btb/ivclsp. FIG. 1 presents for each drug the average value across the cell lines of the TGI, the LC50, and the ratio of LC50 to TGI.

FIG. 2 is a photograph showing a representative example of retinal mounts in the ischemia induced mouse retinopathy model described in Example 1 of mice treated with an intravitreal injection of diluent. The photo demonstrates extensive neovascularization.

FIG. 3 is a photograph showing representative results of photographs of retinal mounts in the ischemia induced mouse retinopathy model described in Example 1 of mice treated with a single intravitreal injection of 0.6 nanogram of bizelesin. The photo (compared to FIG. 2) demonstrates that the bizelesin treatment resulted in nearly complete inhibition of neovascularization.

FIG. 4 shows the average area (and standard deviation) of neovascularization seen with different doses of intravitreal bizelesin in the mouse model of ischemic retinopathy described in Example 1. As indicated by the p values the results were highly statistically significant with a single dose of bizelesin of 0.6 ng and 0.06 ng compared to diluent.

FIG. 5 is a photograph showing a representative results of photographs of retinal mounts of control mice treated with an intravitreal injection of diluent in the laser induced choroidal neovascularization retinopathy model described in Example 2. The photos demonstrate extensive neovascularization (bright green (shown in grayscale in FIGS. 2 and 3 in present application) and arrows) in the area of the laser burn, which is black.

FIG. 6 is a photograph showing representative results of photographs of retinal mounts of mice treated with a single intravitreal injection of 0.6 ng of bizelesin in the laser induced choroidal neovascularization retinopathy model described in Example 2. The photo (compared to FIG. 5) demonstrates nearly complete inhibition of neovascularization in the area of the laser burn.

FIG. 7 is a bar graph showing the average area (and standard deviation) of neovascularization seen with different doses of intravitreal bizelesin and control diluent (in the fellow eyes) in the mouse model of laser induced choroidal neovascularization described in Example 2. As indicated by the p values the results were highly statistically significant with a single dose of bizelesin of 0.06 ng to 6 ng.

### DETAILED DESCRIPTION OF THE INVENTION

A description of example embodiments of the invention follows.

### DEFINITIONS

As used herein, “adduct” refers to a complex formed by covalent linkage or attachment of two molecular entities.

As used herein, “analog” refers to a compound or moiety possessing significant structural similarity as to possess substantially the same function.

As used herein, “AT Islands” refers to Regions of cellular DNA that are enriched in the base sequence adenine, thymine (AT) and which are 50 base pairs or longer; AT islands are critical to cell proliferation.

As used herein, “capsular bag” refers to a sack like bag formed from the fibrous lens capsule after removal of the lens during cataract surgery with phacoemulsification.

As used herein, “clonogenic survival fraction” refers to a measure of to the ability of the cells to proliferate and generate new colonies; the fraction of cells that are able to give rise to a colony of cells in a colony forming assay.

As used herein, “cytotoxic” refers to causing cell death.

As used herein, “derivative” refers to a compound or moiety that has been further modified or functionalized from the corresponding compound or moiety.

As used herein, “gliosis” refers to a proliferation of glial cells.

As used herein, the term “irreversibly inhibit the potential for cell proliferation” refers to permanently inhibit the ability of cells to proliferate; to permanently inhibit clonogenic activity; and to do so without killing nonproliferating cells.

LS50 as used herein refers to the drug concentration that kills 50% of cell.

As used herein, “locally administering” refers to the direct administration of a drug to a site (for example to a target space of the eye), as opposed to drug delivery to that site by a systemic route or intravascular route.

As used herein, “neovacularization of the retina” refers to new blood vessel formulation in or near the retina, reinal neovasculariztion, angiogenesis in the choroid, reina, or epiretinal pace which can include the vitreal space.

As used herein, “nonproliferating cells” refers to cells that are quiescent and not actively engaged in the processes of cellular replication, cells in the G0 stage of the cell cycle or terminally differentiated cells that lack the capacity to replicate.

As used herein, the term “potential for cell proliferation” refers to the ability to proliferate; the ability or potential to form cell colonies; clonogenic potential; the potential for cell proliferation is different from a cell proliferation, cells with the potential for cell proliferation may or may not be engaged in actively proliferating. It is understood that cells in GO can become proliferating cells.

As used herein, “physical carrier” refers to carrier wherein the drug is absorbed on the surface of the physical carrier. Examples of physical carriers include but are not limited to, ophthalmological grade sponges, gauze, cellulose sponges, gels, sutures, plastic membranes, biodegradable and non-biodegradable implants, and intraocular lens implants.

As used herein, the term “prevention” refers to reduction of the risk of developing a particular condition, the act of prophylaxis, for example a reduction of 10%, 20% 30% or 50%.

As used herein, “prodrug” refers to a compound that can undergo bioconversion to the parent drug.

As used herein, “proliferative disorder of the eye” refers to a disorder, disease, or condition characterized by excessive cellular proliferation and growth within the eye that can be non-malignant or malignant; proliferative diseases of the eye.

As used herein, “proliferative retinopathy” refers to a condition, disorder, disease. or process characterized by abnormal cellular growth and proliferation in the retina or adjacent to the retina such as choroidal, epiretinal, or extending into the vitreous; neovascularization or angiogenesis in or near the retina; cell types involve can include: endothelial cells, fibroblasts, RPE cells, glial cells, Mueller cells, astrocytes, fibrocytes, macrophages, inflammatory cells

As used herein “replication fork” refers to site of DNA where the original DNA strands separate to allow DNA synthesis.

As used herein, “target space (e.g., target site)” refers to the anatomical space(s) in the eye within which there is a clinically significant need to prevent or arrest cell proliferation. Target spaces in the eye include but are not limited to pre-retinal, retinal, sub-retinal, scleral, choroidal, vitreal, sub-conjunctival, conjunctival the anterior chamber, posterior chamber, iris, sites of angiogenesis, and at the sites of intraocular tumors and tumor cells. The target space can also be considered as the space into which there is a clinical need to deliver drug so as to practically effect drug delivery to the actual site of pathology. For example, the target space can be the vitreous space for proliferative diseases of the retina and choroid.

As used herein, total growth inhibition, “TGI” refers to the concentration of a drug that causes total growth inhibition of cells, wherein total is defined as about 95% or greater.

As used herein “therapeutic index” refers to the ratio of the drug dose that produces and undesired effect to the dose that produces a desired therapeutic result.

As used herein “toxicity” refers to undesirable or adverse effects, clinically significant side effects or complications

The terms “treating” or “treat” are used interchangeably and include both therapeutic treatment and prophylactic treatment (reducing the likelihood of development or onset, for example, so that onset does not occur, onset is reduced or diminished). Both “treating” or “treat” mean decrease, suppress, attenuate, halt, diminish, arrest, reduce or stabilize the development or progression of a disease, condition or disorder, lessen the severity of the disease, condition or disorder, improve the symptoms associated with the disease, condition or disorder or improve the risk of progression, clinically improve, favorably modify or reduce complications or consequences, or diminish, arrest or lessen the onset of the disease, disorder or condition. Treatment is a means or process for treating a disease, disorder or condition.

The term “effective amount” refers to an amount which, when administered in a proper dosing regime, is sufficient to treat (therapeutically or prophylatically) the target disorder but at a level that is below the concentration or dose required to produce toxicity. For example, an effective amount is sufficient to reduce or ameliorate the progression of the disease or prevent the advancement of the disorder being treated.

The subject typically refers to a human, but can also be an animal, such as companion animals (dogs, cats and the like), farm animals (ruminants, such as cows, pigs, horses, sheep goats and the like) and laboratory animals (such as, rats, mice, guinea pigs and the like).

The present invention relates to a method for the treatment of proliferative eye diseases, including but not limited to diabetic proliferative retinopathy, age related macular degeneration associated proliferative retinopathy, proliferative vitreoretinopathy, epiretinal fibrosis, sub-retinal fibrosis, ocular fibrosis, fibrovascular scarring and gliosis in and near the retina, polypoidal choroidal vasculopathy, epimacular membranes, choroidal neovascularization, retinal angiomatous proliferation, neovascularization of the retina, retinopathy of prematurity, neovascularization related to ocular histoplasmosis, sickle cell proliferative retinopathy, retinal hemagioblastoma in von Hippel-Landau syndrome, pterygia, neovascular glaucoma, iris neovascularization, uveal melanoma, ocular nevi, retinoblastoma, ocular lymphoma, and metastatic cancers to the eye.

The present invention also relates to treating proliferative diseases of the eye in high-risk settings such as reducing the risk of PVR after surgery for retinal detachment or in the early stages of diabetic retinopathy and in the treatment of potentially pre-malignant lesions of the eye to prevent the evolution of intraocular cancers. The potential for cell proliferation is an absolute requirement for the evolution and progression of cancer. Potentially pre-malignant lesions of the eye include but are not limited to dysplastic lesions, pigmented nevi and primary acquired conjunctival melanosis, retinoma. The scope of the present invention also includes the treatment of posterior capsule opacification following cataract extraction and the treatment of scar tissue following glaucoma filtering surgery. The scope of the present invention includes proliferative diseases of the eye in humans and in animal subjects.

The method comprises local delivery of a drug into the target space of the eye, wherein said drug irreversibly inhibits the potential for cell proliferation, and wherein said drug is not cytotoxic to nonproliferating cells. A sufficient quantity or effective amount of drug is delivered to the target space to achieve the desired therapeutic objective. The term “irreversibly inhibits the potential for cell replication” means that the cell permanently loses the capacity to proliferate, in other words clonogenic activity is abolished.

### Drugs for Use in the Methods

A drug suitable for use in the present method has the property that the minimum drug concentration that is generally cytotoxic to cells is substantially higher than the concentration that generally inhibits the potential for cell proliferation. (The term “generally” is used because it is usually possible to develop or find cancer cell lines that are hypersensitive or resistant to any particular agent.) In a preferred embodiment, the drug concentration that generally kills 50% of cells (LD50) is substantially higher than the concentration that totally inhibits cell proliferation (TGI). In a preferred embodiments the ratio of LD50 to TGI is greater then approximately 400, 300, 250, 200, 100, or 50.

In a preferred embodiment the drug is not cytotoxic and permanently abolishes the potential for cell proliferation by irreversibly modifying cells, wherein the actions or consequences of said drug-induced cellular modification are silent, latent, or hidden unless and until the cell enters the proliferative cycle. The term “silent, latent, or hidden” means that there is not significant interference with normal physiological functions of the cell. In a preferred embodiment the drug covalently binds to DNA and interferes with DNA synthesis by stalling replication forks. In a preferred embodiment the drug covalently binds to DNA but does not form adducts with protein or form covalent DNA-drug-protein complexes. In a preferred embodiment the DNA-drug adducts are not repaired by cells or are not excised by cells at a clinically significant rate. Preferably the drug does not induce DNA breaks in non-proliferating cells. Drugs that inhibit the potential for proliferation at picogram/ml to nanogram/ml concentrations and that have the above properties are preferred. An example of such a drug is bizelesin.

In an alternate embodiment, the drug is cytotoxic only to proliferating cells. In this embodiment the drug irreversibly modifies cells, however the action or consequences of said drug-induced cell modification are latent or hidden, until the cell begins the process of replication at which time the drug causes cell death. In a preferred embodiment the drug covalently binds to DNA and interferes with DNA synthesis by stalling replication forks. In a preferred embodiment the drug covalently binds DNA, but does not form adducts with protein or form covalent DNA-drug-protein complexes. In a preferred embodiment the DNA-drug adducts are not repaired by cells or are not excised by cells at a clinically significant rate. Preferably the drug does not induce DNA breaks in non-proliferating cells. Drugs that inhibit the potential for proliferation at picogram/ml to nanogram/ml concentrations and that have the above properties are preferred. An example of such a drug is adozelesin.

In a preferred embodiment the drug used is nontoxic to nonproliferating cells at concentrations that abolish the potential for cell proliferation. In other words, the drug selectively abolishes clonogenic potential without otherwise compromising important physiological cellular functions in non-proliferating cells unrelated to proliferation. In a preferred embodiment the drug covalently binds to cellular components that are critical for proliferation and the potential for proliferation. In a preferred embodiment the drug covalently binds to DNA. In an even more preferred embodiment the drug selectively cross-links to AT islands.

Drugs that covalently bind DNA can cause cellular toxicity and damage. The greater the number of DNA-drug adducts required to inhibit cell proliferation the more extensive the damage and nonspecific impairment of cellular function. Typical DNA alkylating or DNA binding agents require thousands of DNA adducts per cell to inhibit proliferation. For example, approximately 14,000 cisplatin DNA adducts are formed at concentrations that inhibit cell growth by 50%. Woynarowski J M.; Biochim Biophys Acta. 2002 Jul. 18; 1587(2-3):300-8. In preferred embodiments of the present invention a drug is employed that inhibits the potential for cell proliferation (GC50) at low levels of DNA-drug adducts. In preferred embodiments the drug is selected such that the typical number of DNA-Drug adducts required to inhibit cell proliferation 50% is in following approximate ranges: 1 to approximately 10, 10 to approximately 50, 50 to approximately 100, 100 to approximately 500, 500 to approximately 1000. In certain embodiments, preferred are drugs that can inhibit cell proliferation with a GI50 in the picomolar to sub-picomolar range, and for which in clonogenic assays, a plot of the log of the surviving fraction of cells versus the drug concentration is linear or approximately linear. The drugs bizelesin and adozelesin display these properties. When a plot of the log of the surviving fraction of cells versus the drug concentration is linear then the data are consistent with a “single hit” model of inhibition. Lee C S, Gibson N W.; Cancer Res. 1991 Dec. 15; 51(24):6586-91; J F Fowler; 1964 Phys. Med. Biol. 9: 177-188.

Especially preferred drugs are drugs that covalently bind to DNA and that are not excised or removed and that do not evoke a DNA repair response in non-proliferating cells. Typical DNA binding and alkylating agents such as mitomycin-C, cisplatin and temozolomide trigger extensive DNA repair responses. DNA repair responses can be detected by the accumulation of DNA repair proteins such as Replication Protein A (RPA) and histone H2AX phosphorylated at serine 139 (γ-H2AX). Adozelesin is an example of a drug that does not trigger A DNA repair response in non-proliferating cells; Liu J S, Kuo S R, Beerman T A, Melendy T; Mol Cancer Ther. 2003 January; 2(1):41-7. Bizelesin is expected to be similar to adozelesin in this respect as it is forms DNA-drug adducts that are largely hidden within the minor groove of the DNA.

### Delivery of the Drug

The drug are delivered locally into the target site in the eye by means of a local injection, a local infusion, or locally placing a physical carrier that releases the drug. The physical carrier is be impregnated with the drug or coated on the surface with the drug. The physical carrier is used to briefly contact the target site with drug or can be implanted into the eye. The drug is be bound to the physical carrier by noncovalent or by reversible covalent linkages. Reversible covalent linkages that spontaneously release drug are well known to one skilled in the art of medicinal chemistry and prodrug design. In a preferred embodiment the drug is absorbed on the surface of the physical carrier. Examples of physical carriers include but are not limited to, ophthalmological grade sponges, gauze, cellulose sponges, gels, sutures, plastic membranes, biodegradable and non-biodegradable implants, and intraocular lens implants. Biocompatible physical carriers that can be used in the eye are well known to one skilled in the art. See Rautio, J. et al., Nat Rev Drug Discov., 7(3): 255-70 (March 2008); Yasukawa, T. et al., Adv Drug Deliv Rev, 57(14): 2033-46 (Dec. 13, 2005); and Bourges, J. L. et al., Adv Drug Deliv Rev, 58(11):1182-202 (Nov. 15, 2006).

In another embodiment, the drug is infused into the capsular bag following lens extraction in cataract surgery. In a preferred embodiment a device such as the PerfectCapsule® is used to enable drug solution to contact the interior of the capsular bag without contacting other eye structures. After the drug solution has contacted the lens capsule for a sufficient period of time the capsular bag is then rinsed with a solution such as saline to remove the drug solution, prior to removing the irrigation device. In a preferred embodiment the drug concentration is selected such that contact with the drug formulation for at least 5 minutes is required for efficacy. This provides an extra margin of safety. If leakage of the drug should accidentally occur outside of the capsular bag onto the lens or conjunctiva then the area could be irrigated with saline to prevent adverse consequences of drug delivery to unintended sites. A dye can also be added to the visualization of the formulation to enable easier visualization of the drug formulation. Dyes suitable for use in the eye such as sodium fluorescein are well known to one skilled in the art. (Rabsilber, T. M. et al., Br J Ophthalmol, 91(7):912-5 (July 2007)).

### Methods of the Invention

The present invention includes a method for the treatment and prevention of proliferative disorders of the eye comprised of the following steps: selecting a pharmaceutical formulation comprising a drug that irreversibly inhibits the potential for cell replication; defining the target space; and contacting the cells in the target space of the eye with said drug by injecting or infusing it directly into the target space of the eye at a effective amount and for a sufficient period of time to irreversibly inhibit cell proliferation and treat the proliferative disorder; wherein the quantity of said drug is below that required to produce toxicity.

The target space is defined appropriate to the condition being treated using a variety of imaging modalities including direct visualization, ultrasound, angiography, and MRI. In addition, the condition being treated often has a target space that is well known to one skilled in the art and science of ophthalmology. For example, for the treatment of PVR the target space would include the pre-retinal space in the involved region of the eye. The target space in PDR would be the regions of the eye where there is neovascularization or evidence of other abnormal proliferation. The target space for cancers of the eyes would be the volume that has malignant cells and or that has a clinically significant likelihood of containing malignant cells. For POC the target space is the lining of the capsular sac. For pre-malignant nevi the target space is the volume immediately adjacent to or containing the nevi. For the prevention of scarring and fibrosis after glaucoma filtration surgery the target space would be the region of the subconjunctival filtering bleb.

Methods of injection into the eye are well known to one skilled in the art and is done with a needle or catheter inserted into the vitreal space or even more precisely into the desired position(s) with direct visualization with an operative ophthalmologic microscope. Microinfusion techniques, micro-needles and micro-catheters can also be used and are well known to one skilled in the art. A technique for intravitreal injection is provided by the following online video, and is hereby incorporate in its entirety by reference. Folk, J. C.; “Intravitreal Injection Technique” as found on the wide world web at: .medrounds.org/bookstore/ProductDetail.php?product_id=69.

A particular advantage of the present invention is the intraocular delivery of a drug (or set of drugs) that specifically and irreversibly inhibits the potential for cell proliferation without killing nonproliferating cells. The present method has the following major advantages: a) a single brief exposure to the drug will generally be sufficient to inhibit the potential for cell proliferation and treat the proliferative eye disorder; by contrast current therapies require multiple and prolonged dosing regimens; b) broad-spectrum activity; the ability to inhibit essentially all processes that require proliferation including, angiogenesis, fibrosis, gliosis, scar tissue formation, RPE cell proliferation, and the evolution and progression of cancer; c) therapeutic activity regardless of the growth factors that drive cell proliferation in the eye; d) the inhibition of the potential for cell proliferation is independent of the cell cycle; (This is important because only a fraction of malignant cells actively proliferate at a given time); e) absence of cytotoxicity to nonproliferating cells and tissue; (This is in sharp contrast to the prior art, which involves the use of cytotoxic agents) and f) a large therapeutic index, the ratio of LD50 to TGI is large.

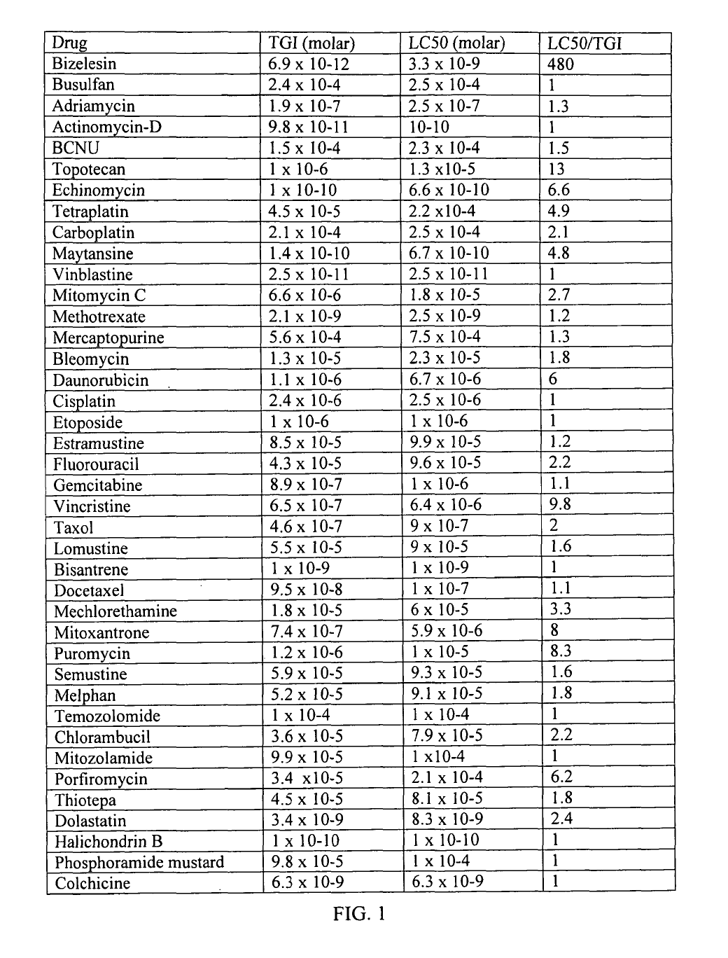

Conventional anti-proliferative drugs such as adriamycin, cisplatin, taxol, vincristine, mitomycin, busulfan, Actinomycin-D, and phosphoramide mustards are highly cytotoxic, kill proliferating as well as nonproliferating cells, and cause tissue damage after local administration. In addition such agents damage cells and can impair cell function. Cytostatic agents generally require continuous drug exposure for continuous or inhibition of cell proliferation. For typical antiproliferative agents the concentration required to inhibit cell proliferation is comparable to that which is cytotoxic to cells. (See FIG. 1).

In a preferred embodiment the drug is bizelesin. In another preferred embodiment the drug is adozelesin. In a preferred embodiment one or more drugs is selected from the following group: adozelesin; bizelesin; U77809, U78779, carzelesin; Brostallicin; tallimustine, or derivatives, prodrugs, analogues, and active metabolites thereof. These drugs covalently bind to the minor groove of DNA in adenine-thymine rich regions and inhibit cell proliferation at sub-nanomolar concentrations. Bizelesin; and its active metabolite U77809 cross-link adjacent DNA strands. One skilled in the art can readily identify using routine and well-known experimental techniques additional drugs that can irreversibly and permanently arrest the potential for cell replication without causing extensive cell death or tissue necrosis upon local injection. Such drugs are to be within the scope of the present methods and invention.

The following references relate to this matter: Li, L. H. et al., Invest New Drugs, 9(2): 137-48 (May 1991); Liu, J. S. et al., Mol Cancer Ther, 2(1): 41-7 (January 2003); Liu, J. S. et al., Mutat Res, 532(1-2): 215-26 (Nov. 27, 2003); Liu, J. S. et al., J Biol Chem, 275(2): 1391-7 (Jan. 14, 2000); Cao, P. R. et al., Mol Cancer Ther, 2(7): 651-9 (July 2003); Hess, M. T. et al., Nucleic Acids Res, 24(5): 824-8 (Mar. 1, 1996); Cristofanilli, M. et al., Anticancer Drugs, 9(9): 779-82 (October 1998); Burris, H. A. et al., Anticancer Drugs, 8(6): 588-96 (July 1997); Liu, J. S. et al., J Biol Chem, 275(2): 1391-7 (Jan. 14, 2000); Bhuyan, B. K. et al., Cancer Chemother Pharmacol, 30(5): 348-54 (1992); Herzig, M. C. et al., Biochemistry, 38(42):14045-55 (Oct. 19, 1999); Schwartz, G. H. et al., Ann Oncol, 14(5): 775-82 (May 2003); Pepper, C. J. et al., Cancer Res, 64(18): 6750-5 (Sep. 15, 2004); Alley, M. C. et al., Cancer Res, 64(18): 6700-6 (Sep. 15, 2004); Hartley, J. A. et al. Cancer Res, 64(18): 6693-9 (Sep. 15, 2004); Pavlidis, N. et al., Cancer Chemother Pharmacol, 46(2): 167-71 (2000); Awada, A. et al., Br J Cancer, 79(9-10): 1454-61 (March 1999); Gregson, S. J. et al., J Med Chem, 47(5): 1161-74 (Feb. 26, 2004); Gregson, S. J. et al., J Med Chem, 44(5): 737-48 (Mar. 1, 2001); Li, L. H. et al., Cancer Res, 52(18): 4904-13 (Sep. 15, 1992); Woynarowski, J. M., Curr Cancer Drug Targets, 4(2): 219 (March 2004); Woynarowski, J. M. et al., J Biol Chem, 276(44): 40555-66 (Nov. 2, 2001); Woynarowski, J. M., Biochim Biophys Acta, 1587(2-3): 300-8 (Jul. 18, 2002); Lockhart, A. C. et al., Clin Cancer Res, 10(2): 468-75 (Jan. 15, 2004); Rossi, R. et al., Anticancer Res, 16(6B): 3779-83 (November-December 1996); Cozzi, P., Farmaco, 55(3): 168-73 (March 2000); Rajski, S. R. et al., Chem Rev, 98(8):2723-2796 (Dec. 17, 1998).

In a preferred embodiment the drug selected binds in the minor grove of DNA and crosslinks adjacent strands. In a preferred embodiment the drug selectively crosslinks AT islands of cellular DNA. AT-Rich islands are critical to cell proliferation. In a preferred embodiment the crosslinks are not excised or repaired by cells. In a preferred embodiment of the present invention two drugs each capable of irreversibly inhibiting cell proliferation are co-administered. In a preferred embodiment one of the drugs covalently binds to A-T regions of DNA and the other covalently binds to guanine regions. In a preferred embodiment these drugs cross-link the DNA. In a preferred embodiment the drug covalently cross-links adjacent strands of DNA. In a preferred embodiment one drug is selected from the following group: adozelesin; bizelesin; U77809, U78779, carzelesin; Brostallicin; tallimustine; or derivatives, analogues, and active metabolites thereof.

The above referenced drugs are toxic with dose limiting bone marrow toxicity following systemic administration. It is critical that the total dose of drug administered be significantly less that that which produces systemic toxicity. As a safe guard the drugs should be pre-packaged in systemically nontoxic quantities. Following local administration into the eye the drugs will be rapidly taken up by cells and irreversibly bound to the cellular DNA. The hydrophobic nature of the drugs will strongly favor local uptake into the tissue. Accordingly, the local concentration of drug achieved within the eye can be orders of magnitude higher than systemic levels. This can translate into enormously increased local inhibition of the potential for cell replication compared to that at distant sites. For many drugs, including Bizelesin there is an linear relationship between the log of the clonogenic cell survival fraction and the drug concentration. Additive increases in drug concentration can give exponential decreases in the probability of clonogenic cell survival. The following reference relates to this matter: Brown J M, Wouters B G.; Cancer Res. 1999 Apr. 1; 59(7):1391-9. Lee C S, Gibson N W. Cancer Res. December 15; 51(24):6586-91 (1991).

For optimal therapeutic benefit in some conditions, such as ocular cancer, it is important that the drugs broadly contact cells within the target volume of the eye. This can be achieved by injecting or infusing the drug into the target volume of the eye at multiple locations either with multiple needles or catheters or by repositioning one needle as needed. The needle or catheter can have multiple holes to aid in drug dispersion. In a preferred embodiment the drug is co-infused with a diagnostic imaging agent to enable the volume that has been injected to be visualized. A variety of suitable imaging agents and dyes are well known to one skilled in the art that can be used to visualize injections in the eye. Although a single treatment will generally suffice, the treatment can be repeated if needed to achieve the desired clinical result.

In a preferred embodiment the drug is bizelesin. Bizelesin has a number of unexpected properties that make it highly suited for treating proliferative disorders of the eye. The drug is an ultra-potent irreversible inhibitor of cell proliferation. As few as 1 to approximately 10 bizelesin-DNA crosslinks can inhibit cell proliferation. The small number of drug molecules per cell needed to inhibit clonogenic potential limits collateral damage to cellular processes in nonproliferating cells. Bizelesin is rapidly and irreversibly retained by cells and has poor systemic bioavailability unless given intravascularly. There is a large difference between the bizelesin concentration that inhibits cell proliferation and the concentration that kills cells. This unexpected property is in sharp contrast to typical anticancer drugs as evidence by the data shown in FIG. 1, which summarizes screening results obtained with forty different anticancer agents in the National Cancer Institute (NCI) DTP Human Tumor Cell Line Screen. In this assay approximately 60 different human cancer cell lines were incubated for 48 hours with anticancer drugs. A colorimetric assay was employed to enable the calculation of the drug concentration that completely inhibited cell growth (TGI) and the drug concentration that resulted in 50% cell killing (LC50). Details of the assay are available on the world wide web at :dtp.nci.nih.gov/branches/btb/ivclsp.

FIG. 1 presents for each drug the average value across the cell lines of the TGI, the LC50, and the ratio of LC50 to TGI. The data show that Bizelesin is in a class by itself with an extraordinarily large LC50 to TGI ratio of 480 and a TGI in the picomolar range. (It should be noted that other data sets for Bizelesin failed to show picomolar drug potency due to the inadequate manner in which the test solutions were prepared and precipitation/absorption of the drug. (Personal communication Dr. Jan. Woynarowski, NCI) The remarkable difference between the concentration of Bizelesin that inhibits clonogenic activity and the concentration that is cytotoxic is further illustrated by experiments with HCT116 colon cancer cells. Treatment with 250 times the GC50 concentration of bizelesin for 4 days did not cause apoptosis. The following reference relates to this matter: Cao P R, McHugh M M, Melendy T, Beerman T; Mol Cancer Ther. July; 2(7):651-9 (2003).

In a preferred embodiment the bizelesin dose is delivered by intravitreal injection; wherein said dose is selected from the following approximate dose ranges: 001 pg to 1 pg, 0.001 ng to 0.01 ng, 0.01 ng to 0.1 ng, 0.1 ng to 1 ng, 1 ng to 5 ng, 5 ng to 10 ng, 10 ng to 20 ng, 20 ng to 50 ng, 50 ng to 250 ng. In certain embodiments, the dose is 0.001 ng, 0.01 ng, 0.02 ng, 0.03 ng, 0.04 ng, 0.05 ng, 0.06 ng, 0.07 ng, 0.08 ng, 0.09 ng, 0.1 ng, 0.2 ng, 0.3 ng, 0.4 ng, 0.5 ng, 0.6 ng, 0.7 ng, 0.8 ng, 0.9 ng, 1 ng, 2 ng, 3 ng, 4 ng, 5 ng, 6 ng, 7 ng, 8 ng, 9 ng, 10 ng.

In a preferred embodiment a single intravitreal administration of drug is given into the eye. In other embodiments the intravitreal injection is repeated at weekly, monthly, or every 2, 3, 4, 5, 6, 7, 8, 9, 10, 11, 12, 18, 24, 36, or 48 months, or given as clinically indicated.

In preferred embodiments an intravitreal injection of bizelesin is used to treat a proliferative eye disorder selected from the following list: diabetic proliferative retinopathy, age related macular degeneration associated proliferative retinopathy, proliferative vitreoretinopathy, epiretinal fibrosis, sub-retinal fibrosis, ocular fibrosis, fibrovascular scarring and gliosis in and near the retina, polypoidal choroidal vasculopathy, epimacular membranes choroidal neovascularization, retinal angiomatous proliferation, neovascularization of the retina, retinopathy of prematurity, neovascularization related to ocular histoplasmosis, retinal hemagioblastoma in von Hippel-Landau syndrome, uveal melanoma, ocular nevi, retinoblastoma, ocular lymphoma, and metastatic cancers to the eye. The means to diagnose these conditions are well known to one skilled in the art.

In a preferred embodiments an intravitreal injection of bizelesin is used to treat and reduce the risk of developing a proliferative eye disorder selected from the following list: diabetic proliferative retinopathy, age related macular degeneration associated proliferative retinopathy, proliferative vitreoretinopathy; the progression of potentially pre-malignant ocular lesions into malignant lesions.

In a preferred embodiment the proliferative eye disorder is posterior capsular opacification following cataract surgery, the target space is the lining or interior surface of the capsular bag, and the drug is Bizelesin. In a preferred embodiment the drug is infused into the capsular bag using a sealed system such as the PerfectCapsule®. At the completion of the treatment the capsular bag is irrigated with a solution such as saline to remove excess drug, prior to removal of the delivery device. Methods for using such closed irrigation systems such as the PerfectCapsule® are well known to one skilled in the art.

In another preferred embodiment the Bizelesin is absorbed onto the surface of the implanted lens.

In another preferred embodiment the drug is adozelesin. Adozelesin irreversibly binds to the minor grove of DNA and is highly toxic for proliferating cells. Adozelesin may be likened to a hidden time bomb waiting to go off when the cell enters S-phase to proliferate. The following references relate to this matter: Liu J S, Kuo S R, Beerman T A, Melendy T.; Mol Cancer Ther. 2003 January; 2(1):41-7; Bhuyan B K, Smith K S, Adams E G, Wallace T L, Von Hoff D D, Li L H.; Cancer Chemother Pharmacol. 1992; 30(5):348-54.

In a preferred embodiment the adozelesin dose is delivered by intravitreal injection; wherein said dose is selected from the following dose ranges 0.001 pg to 1 pg, 0.01 ng to 0.1 ng, 0.1 ng to 1 ng, 1 ng to 5 ng, 5 ng to 10 ng, 10 ng-20 ng, 20 ng to 50 ng, 50 ng to 250 ng and 250 ng to 2000 ng. In a preferred embodiment a single intravitreal administration of drug is given into the eye. In other embodiments the intravitreal injection is repeated at weekly, monthly, or every 2, 3, 4, 5, 6, 7, 8, 9, 10, 11, 12, 18, 24, 36, or 48 months or given as clinically indicated.

In a preferred embodiment an intravitreal injection of adozelesin is used to treat a proliferative eye disorder selected from the following list: diabetic proliferative retinopathy, age related macular degeneration associated proliferative retinopathy, proliferative vitreoretinopathy, epiretinal fibrosis, sub-retinal fibrosis, ocular fibrosis, fibrovascular scarring and gliosis in and near the retina, polypoidal choroidal vasculopathy, epimacular membranes choroidal neovascularization, retinal angiomatous proliferation, neovascularization of the retina, retinopathy of prematurity, neovascularization related to ocular histoplasmosis, retinal hemagioblastoma in von Hippel-Landau syndrome, uveal melanoma, ocular nevi, retinoblastoma, ocular lymphoma, and metastatic cancers to the eye.

In a preferred embodiments an intravitreal injection of adozelesin is used to treat and reduce the risk of developing a proliferative eye disorder selected from the following list: diabetic proliferative retinopathy, age related macular degeneration associated proliferative retinopathy, proliferative vitreoretinopathy; the progression of potentially pre-malignant ocular lesions into malignant lesions.

In a preferred embodiment the proliferative eye disorder is posterior capsular opacification following cataract surgery, the target space is the lining or interior surface of the capsular bag, and the drug is adozelesin. In a preferred embodiment the drug is infused into the capsular bag using a sealed system such as the PerfectCapsule®. At the completion of the treatment the capsular bag is irrigated with a solution such as saline to remove excess drug, prior to removal of the delivery device. In another preferred embodiment the adozelesin is absorbed onto the surface of the implanted lens.

In a preferred embodiment the method is used to treat scarring and fibrosis following glaucoma filtration surgery. bizelesin is the preferred drug and it is administered locally to the site of the subconjunctival drainage bleb by means of injection or by briefly contacting the site with bizelesin on a physical carrier such as an ophthalmological surgical sponge. Such methods of administration are well known to one skilled in the art and are the same as those commonly used to administer 5 fluorouracil or mitomycin-C to reduce scarring for the same condition. The dose of bizelesin administered for this indication is preferably in the range of 1 picogram to 1 nanogram. An example of a suitable technique for administering the drug is provided in the following reference videos. Kwon, Y. H.; “Combined Mitomycin C Trabeculectomy and Trabeculotomy in Congenital Glaucoma”, available on line at medrounds.org/bookstore/ProductDetail.php?product_id=121&PHPSESSID=df58dc22b631b28392395eacdf952917 and in Kwon, Y. H.; “Combined Mitomycin C Trabeculectomy for Treatment of Glaucoma”; which is available on line at.medrounds.org/bookstore/ProductDetail.php?product_id=122, and in the DVD, “Master Techniques in Glaucoma Surgery”, by Young H. Kwon, (2006), MedRounds Publications, Inc., which is hereby incorporated in its entirety by reference. In an alternate embodiment adozelesin is employed in place of the bizelesin to treat scarring and fibrosis following glaucoma filtration surgery.

In an alternate embodiment bizelesin and adozelesin are used in combination for the treating proliferative diseases of the eye.

The scope of the present invention includes a kit for intraocular therapy of proliferative eye disorders comprised of sterile, pyrogen free drug and diluent packaged together as single dose unit. The kit is used by dispensing the diluent into the Bizelesin container, mixing to dissolve the drug, aspirating the drug formulation into the syringe, and proceeding to administer the indicated volume of drug from the syringe into the patient by means well known to one skilled in the art and science of ophthalmology.

In a preferred embodiment the drug in the kit is bizelesin. In a preferred embodiment the sterile kit is comprised of a container with Bizelesin dissolved in an non-aqueous frozen solvent, preferably under a dry inert atmosphere such as nitrogen or argon, and a second container with a buffered saline diluent, which can contain other excipients. Immediately prior to use the Bizelesin solution is thawed and diluted with the diluent. In another preferred embodiment the kit also includes a syringe and needles for mixing the diluent and drug and a second needle for the intraocular injection; wherein the syringe, needle, and drug concentration and formulation are selected such that the desired therapeutic dose of drug is actually delivered out of the needle tip. This is important because for Bizelesin the total drug dose will be in the range of approximately 100 picograms to 50 nanograms. A standardized kit is needed to address the issue of drug absorption to the walls of the syringe, containers, and needle and enable reliable drug dosing.

The present invention also includes a kit as described above in which the Bizelesin is prepackaged in the vial as an inclusion complex with cyclodextrin, rather than dissolved in an organic solvent. Preferably an excess of the cyclodextrin is used.

The present invention also includes an implantable lens to which is reversibly absorbed a drug that can irreversibly inhibit the potential for cell proliferation. In a preferred embodiment the drug is Bizelesin. In another preferred embodiment the drug is adozelesin. Such lens can be prepared by contacting the lens with a solution containing the desired dose of the drug and removing the solvent.

The present invention also includes a kit for the prevention of posterior capsular opacification consisting of a sterile intraocular lens with a single dose of a drug that can irreversibly inhibit the potential for cell proliferation onto its surface. In a preferred embodiment the drug is bizelesin.

In certain embodiments, a method for treating proliferative eye diseases comprised of the injection of Bizelesin into the eye is described. In other embodiments, a method for treating proliferative eye diseases, including retinal neovascularization, choroidal neovascularization age related macular degeneration associated proliferative retinopathy, diabetic proliferative retinopathy, proliferative vitreoretinopathy, ocular fibrosis, fibrovascular scarring and gliosis, ocular cancer, posterior capsular opacification comprised of the intravitreal injection of Bizelesin into the eye is described. In another embodiment, a method for treating fibrosis and scarring after glaucoma filtration surgery comprised of the administration of Bizelesin into the site of the subconjunctival drainage bleb is described.

In another aspect of the invention, a single dose kit comprised of the drug Bizelesin formulated for injection into the eye is described.

In other embodiments, methods for treating proliferative eye diseases, for example choroidal neovascularization, age related macular degeneration associated proliferative retinopathy, diabetic proliferative retinopathy, proliferative vitreoretinopathy, ocular fibrosis, fibrovascular scarring and gliosis, ocular cancer, posterior capsular opacification comprising local administration of adozelesin into the eye is described. In certain aspects, the administration is by intravitreal injection of adozelesin into the eye. In certain aspects the drug in injected into the capsular bag. In other aspects, a method for treating fibrosis and scarring after glaucoma filtration surgery comprised of the administration of adozelesin into the site of the subconjunctival drainage bleb is described. In another aspect, a single dose kit comprised of the drug adozelesin formulated for injection into the eye is described.

### Formulations for the Methods

The drug can be formulated in a pharmaceutical composition comprising an effective amount of a drug, or a pharmaceutically acceptable salt of said drug and a pharmaceutically acceptable carrier. The carriers are “pharmaceutically acceptable” in that they are not deleterious to the recipient thereof in an amount used in the medicament. Pharmaceutically acceptable carriers, adjuvants and vehicles that may be used in the methods of this invention include, but are not limited to, ion exchangers, lecithin, serum proteins (e.g., human serum albumin), buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The formulation can be a solution, suspension, emulsion, gel, polymeric paste, nanoparticles, microspheres, or liposomal preparation. The drugs can be administered in combination with commonly employed pharmacological excipients, which include but are not limited to, saline, aqueous buffers, dimethylsulfoxide, dimethylforamide, dimethylacetamide, N-methyl-2-pyrrolidone, cyclodextrins, sodium hyaluronate, emulsifying agents, preservatives and stabilizers that are well known to one skilled in the art. The drug can be dissolved in sterile saline or water or a buffered salt solution. In a preferred embodiment the drug is dissolved in an ophthalmological formulation of 1% sodium hyaluronate. In a preferred embodiment no organic solvent is employed in the formulation, rather the drug is formulated as a dry cyclodextrin inclusion complex, to which is added an aqueous solution such as saline, or ophthalmological grade of 1% sodium hyaluronate prior to administration. In a preferred embodiment the cyclodextrin is hydroxypropyl-β-cyclodextrin such as KLEPTOSE HPB®. Techniques for the formation of drug-cyclodextrin inclusion complexes and the pharmaceutical uses of cyclodextrins are well known to one skilled in the art. The following reference relates to this matter and is herby incorporated by reference. Challa, R. et al., AAPS PharmSciTech, 6(2): E329-57 (Oct. 14, 2005); Rajewski, R. A. et al., J Pharm Sci, 85(11): 1142-69 (November 1996); and Afzal, A. et al., Brain Res Bull, (Aug. 11, 2009).

The concentration, volume and total dose of the drug is dependent upon the clinical condition to be treated and the desired pharmacological effect. For intravitreal injections the injection volume will typically be in the range of 1 microliter to 100 microliters. Even smaller volumes can be used if needed for direct intra-lesional therapy. In a preferred embodiment the injection volume is 50 microliters for intravitreal injections. The drug dose will generally be less than 10% of that which can produce systemic toxicity such as decreased white blood cell count, and preferably less than 1% of said dose. Sub-nanogram to microgram quantities should be sufficient (depending upon the particular agent) because of the extreme potency of the drugs. Techniques for the determination of clinical drug doses and concentrations are well known to one skilled in the art. The drug may be administered by one or more local injections or by catheters placed within the eye and connected to microinfusion pumps. Suitable catheters and micro-infusion pumps, and injection techniques are well known to one skilled in the art.

### EXAMPLES

### Example 1

Intravitreal bizelesin was evaluated in the mouse model of ischemic proliferative retinopathy. Details of the model are provided in Xie, B. et al., J Cell Physiol, 218(1): 192-8 (January 2009). The animal work was done in accordance with the Association for Research in Vision and Ophthalmology Statement for the Use of Animals in Ophthalmic and Vision Research and the guidelines of the Animal Care and Use Committee.

Bizelesin was dissolved in DMSO PharmaSolvent (Gaylord Chemical, Inc.) at 20 microgram/ml and filtered sterilized with a 0.2 micron Milliex-LG Millipore filter. HPLC analysis of the DMSO drug solution post filtration revealed that the bizelesin concentration was 11 microgram/ml. The bizelesin solution was stored at −65 C or below. Immediately prior to administration the drug solution was thawed and diluted with sterile phosphate buffered saline (PBS).

In brief, for the ischemic proliferative retinopathy model, liters of C57BL/6 mice were exposed to 75% oxygen from P7 to P12 (P=age in days) and then returned to room air. On P12 the mice were anesthetized and given an intravitreal injection of 1 microliter of bizelesin solution or 1 microliter of control diluent. The dose of bizelesin ranged from 0.6 picogram to 0.6 nanograms. On the evening of P16 the mice were given an intravitreal injection of anti-mouse platelet endothelial cell adhesion molecule-1 (PECAM-1) antibody, which stain new blood vessels. On P17, the retinas were isolated, flat mounts prepared, and the area of neovascularization was determined with fluorescent microscopy and computerized image analysis.

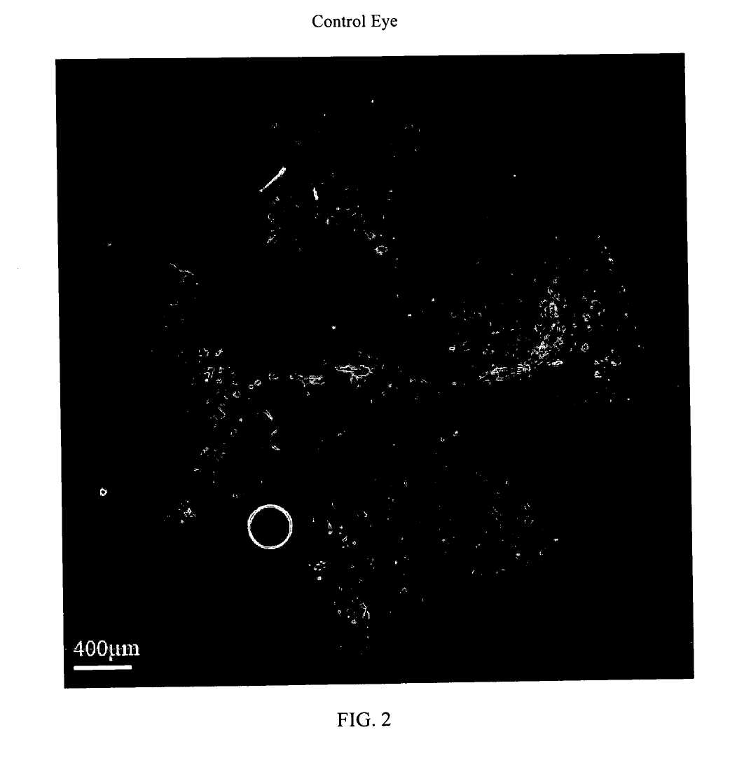

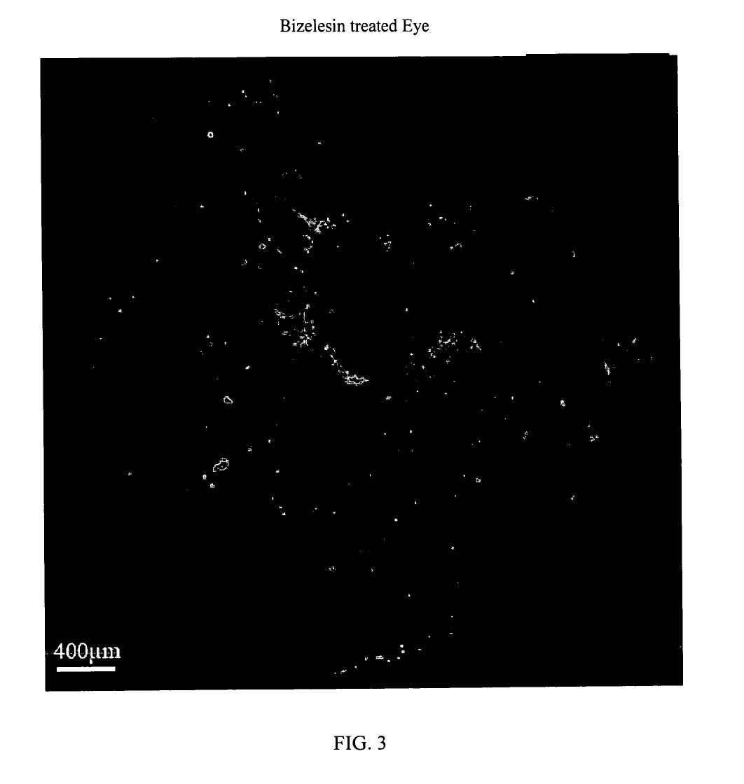

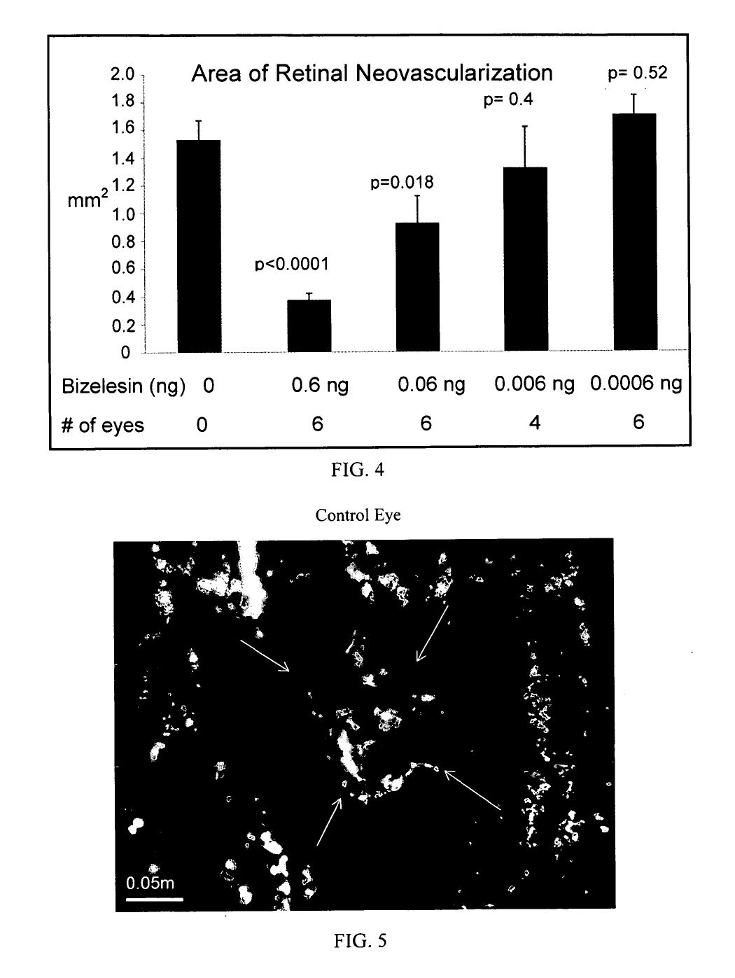

Eyes treated with control diluent developed extensive retinal neovascularization. By contrast, bizelesin treatment at a dose of only 0.6 nanogram resulted in nearly complete inhibition of the proliferative retinopathy and neovascularization without any clinical evidence of ocular toxicity. Representative results are shown in FIG. 2 and FIG. 3. The dose response data are summarized in FIG. 4.

### Example 2

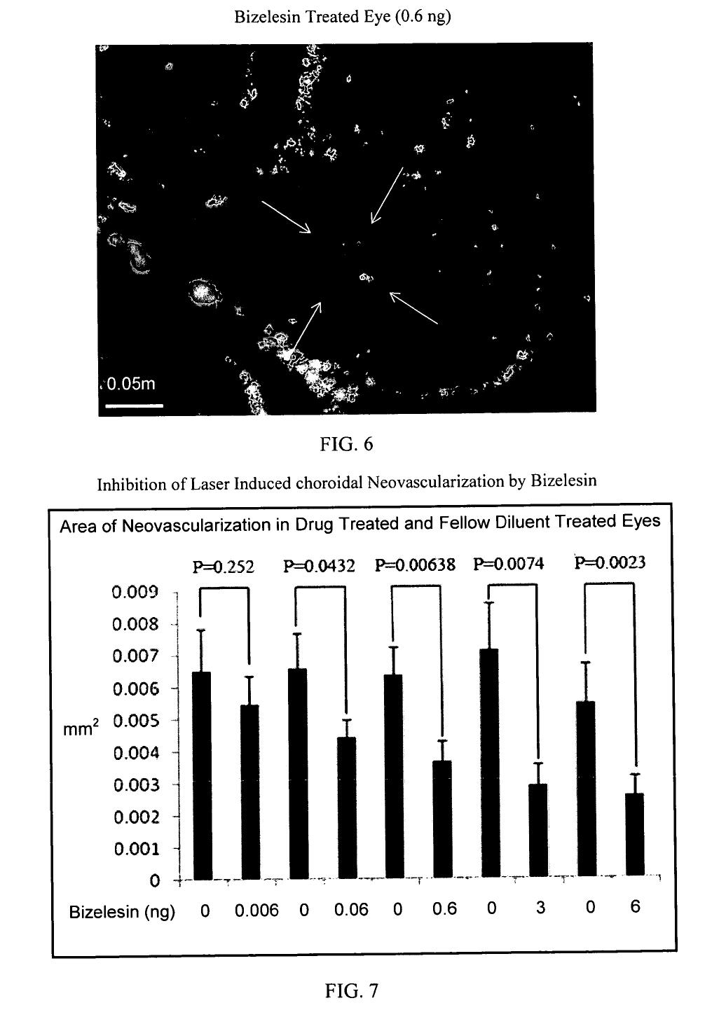

Intravitreal bizelesin was evaluated in the laser induced mouse model of choroidal neovascularization. Details of the model are provided in Xie, B. et al., “Blockade of Sphingosine-1-phosphate Reduces Macrophage Influx and Retinal and Choroidal Neovascularization,” J Cell Physiol, 218(1): 192-8 (January 2009 Bizelesin was formulated in PBS as described in Example 1. In brief, 5-6 week old C57BL/6 mice on day 0 were anesthetized and choroidal neovascularization was induced by laser photocoagulation-induced rupture of Bruch's membrane. The mice were then given an intravitreal injection of 1 microliter of control diluent in one eye and in the contralateral fellow eye 1 microliter of solution containing 6 ng to 0.006 ng of Bizelesin. Ten mice were employed per dose level. On day 14 the mice were perfused with fluorescein-labeled dextran, the retinas were isolated, flat mounts prepared, and the area of neovascularization was determined with fluorescent microscopy and computerized image analysis. FIG. 5 shows representative results of the extensive neovascularization seen in control, diluent treated eyes. Treatment with a single intravitreal dose of 0.6 ng of Bizelesin resulted in dramatic inhibition of the neovascularization. (FIG. 6). The dose response data are shown in FIG. 7 and demonstrate highly potent and statistically significant inhibition of choroidal neovascularization after a single dose of Bizelesin of 0.06 ng to 6 ng. There was no clinical evidence of ocular toxicity from the intravitreal Bizelesin.

### Example 3

This is an example of a single dose kit for intravitreal injection of bizelesin for the treatment of proliferative eye disorders including but not limited to diabetic proliferative retinopathy, age related macular degeneration associated proliferative retinopathy, proliferative vitreoretinopathy, sub-retinal fibrosis, polypoidal choroidal vasculopathy, proliferative vitreoretinopathy, epimacular membranes choroidal neovascularization, neovascularization of the retina, retinopathy of prematurity, neovascularization related to ocular histoplasmosis, retinal hemagioblastoma in von Hippel-Landau syndrome, uveal melanoma, ocular nevi, retinoblastoma, ocular lymphoma, and metastatic cancers to the eye.

The kit consists of:

A sterile solution of bizelesin (0.05 ng to 100 ng) dissolved in 10 to 50 microliters of pharmaceutical grade, anhydrous, dimethylsulfoxide in a labeled, amber glass vial, with a Teflon coated rubber septum, filled with dry nitrogen. The vial is stored at −20 C or below.

A sterile, labeled vial of pharmaceutical grade normal saline, 1.5 ml

A sterile calibrated 1 ml glass syringe with a 5-micron filtered 19-gauge needle

A sterile 30 gauge ½ inch needle

A sterile glass vial with a screw cap for disposal of excess drug

The kit is used by:

Thawing the frozen bizelesin solution

Using the 19-gauge needle and 1 ml glass syringe to dispense 1.0 ml of saline into bizelesin the vial

Mixing the bizelesin by aspirating back and forth into the syringe

Drawing up 0.75 ml of drug solution into the syringe with the 19-gauge needle

Removing the 19 gauge needle and replacing it with the 30-gauge needle

Expelling all but the desired injection volume (typically 50 to 100 microliters) from the syringe into the sterile glass waste vial

Wiping the surface of the 30-gauge needle against sterile gauze to remove any fluid on the needle surface

Injecting the contents of the syringe into the vitreous of the eye using techniques well known to one skilled in the art and science of ophthalmology

### Example 4

In place of DMSO in Example 3 the solvent employed is pharmaceutical grade, anhydrous N,N-dimethylacetamide.

### Example 5

In place of DMSO in Example 3 the solvent employed is pharmaceutical grade, anhydrous N,N,-dimethylforamide

### Example 6

In place of DMSO in Example 3 the solvent employed is pharmaceutical grade, anhydrous N-methyl-2-pyrrolidone.

### Example 7

In place of DMSO in Example 3 no organic solvent is employed. The bizelesin is formulated as a dry inclusion complex in sterile pharmaceutical grade, hydroxypropyl-β-cyclodextrin. The quantity of cyclodextrin is selected to be in the range of 0.05 mg to 10 mg.

### EQUIVALENTS

Those skilled in the art can recognize or be able to ascertain, using no more then routine experimentation, many equivalents to the inventions, materials, methods, and components described herein. Such equivalents are intended to be within the scope of the claims of this patent. While this invention has been particularly shown and described with references to preferred embodiments thereof, it will be understood by those skilled in the art that various changes in form and details may be made therein without departing from the scope of the present invention.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A method for treatment of a proliferative disease, disorder or condition of the eye, comprising locally administering bizelesin or adozelesin into the target space of the eye, wherein said bizelesin or adozelesin irreversibly inhibits the potential for cell proliferation, and wherein said bizelesin or adozelesin is not cytotoxic to nonproliferating cells.

2. (canceled)

3. The method of claim 1, wherein the proliferative eye disorder is selected from the group consisting of: diabetic proliferative retinopathy, age related macular degeneration, associated proliferative retinopathy, proliferative vitreoretinopathy, sub-retinal fibrosis, polypoidal choroidal vasculopathy, proliferative vitreoretinopathy, epimacular membranes, choroidal neovascularization, neovascularization of the retina, retinopathy of prematurity, neovascularization related to ocular histoplasmosis, retinal hemagioblastoma in von Hippel-Landau syndrome, scarring after glaucoma filtration surgery, uveal melanoma, ocular nevi, retinoblastoma, ocular lymphoma, metastatic cancers to the eye, pre-malignant lesions of the eye dysplastic lesions, pigmented nevi and primary acquired conjunctival melanosis.

4. The method of claim 1, wherein the condition is neovascularization of the retina or choroidal neovascularization.

5. The method of claim 1, wherein the drug is administered as an intravitreal injection.

6. The method of claim 1, wherein the condition is diabetic proliferative retinopathy.

7. The method of claim 1, wherein the condition is age related macular degeneration associated proliferative retinopathy.

8. The method of claim 1, wherein the condition is proliferative vitreoretinopathy.

9. (canceled)

10. The method of claim 1, wherein:
the condition is posterior capsular opacification; and
wherein the bizelesin is administered into the capsular bag at the time of cataract surgery.

11. The method of claim 1, wherein the dose of bizelesin or adozelesin is in the range of 0.0001 ng to 100.0 ng.

12-14. (canceled)

15. A method for the prevention of posterior capsule opacification following cataract extraction comprising the following steps:
i.) selecting a pharmaceutical formulation comprising a drug that irreversibly inhibits the potential for cell replication;
ii.) contacting cells in the posterior capsule of the lens with said drug at an effective amount for a sufficient period of time to locally abolish the potential for cell proliferation; wherein the quantity of said drug is at dose below that required to produce toxicity.

16. The method of claim 15, wherein the drug is bizelesin.

17. The method of claim 16, wherein the posterior capsule of the lens is contacted with the bizelesin by means of a physical carrier impregnated with the drug or with the drug absorbed on the surface of the physical carrier.

18. The method of claim 17, wherein the physical carrier is an implantable lens.

19. A method for the treatment of proliferative eye disorders comprising the following steps:
i.) selecting a pharmaceutical formulation comprising a drug that irreversibly inhibits the potential for cell replication;
ii.) defining a target space; and
iii) contacting cells in the target space of the eye with said drug by injecting or infusing the drug directly into the target space of the eye at an effective amount for a sufficient period of time to treat the proliferative disorder; and wherein the quantity of said drug is at dose below that required to produce toxicity.

20. The method of claim 19, wherein the drug is bizelesin or adozelesin.

21. The method of claim 16, wherein the dose is in the range of 0.0001 ng to 100.0 ng.

22. The method of claim 19, wherein the dose is in the range of 0.0001 ng to 100.0 ng.
